SEQUENCE LISTING (10 sequences)

SEQ ID NO: 1 - DNA, 27 bp, homo sapien
tcgcaggcactattctaactactttac

SEQ ID NO: 2 - DNA, 26 bp, homo sapien
gcattcctgaaaagagagatcatgtg

SEQ ID NO: 3 - DNA, 25 bp, homo sapien
gctacagagcagacagttaagagaa

SEQ ID NO: 4 - DNA, 22 bp, homo sapien
tcatggagtgcagctcatttct

SEQ ID NO: 5 - DNA, 21 bp, homo sapien
cttgagcccagagtgaacact

SEQ ID NO: 6 - DNA, 25 bp, homo sapien
accctcatccctggagttttataca

SEQ ID NO: 7 - DNA, 6029 bp, homo sapien
atgaagcccagagcggagtgctgttctcccaagttctggttggtgttggccgtcctggcggtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagccccctgccaaaaacgcatagtcactgagaataagacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccgtcagcggaggagggccctgtaccaacaggtcccatatcaagcacgggacgggcgacaaacacggcgtggtcagcggggtacctgcaccttgggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaactgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctgagtgagggaacagagaggttaaggtgggtacgggagggtaaggctgtgggtcgcgtgatgcgcatgtcacggagggtgacgggggtgaacttggttcccatttgctcctttcttgttttaatttatttatggggatcctggagttctggttcctactgggggcaaccctggtgaccagcaccatctctcctccttttcacagttctctccttcttccccccgctctcagccattcctgttcccatgagatgatgccatgggtctcagcaggggagggtagagcggagaaaggaagggcagcatgcgggcttcctcctggtgtggaagagctccttgatatcctctttgagtgaagctgggagaaccaaaaagaggctatgtgagcacaaaggtagcttttcccaaactgatcttttcatttaggtgaggaagcaaaagcatctatgtgagaccatttagcacactgcttgtgaaaggaaagaggctctggctaaattcatgctgcttagatgacatctgtctaggaatcatggtccaagcagaggttgggaggccatttgtgtttatatataagccaaaaaatgcttgcttcaaccccatgagactcgatagtggtggtgaacagaacaaaaggtcattggtggcagagtggattcttgaacaaactggaaagtacgttatgatagtgtcccacggtgccttggggacaagagcaggtggattgtgcgtgcatgtgtgttcatgcacacttgcacccatgtgtagtcaggtgcctcaagagaaggcaaccttgactctttctattgtttctttcaatatccccaagcagtgtgattgtttggcttatatacagacagagatggccatgtattacctgaattttggctgtgtctcccttcatccttctggaataaggagaatgaaaattcttgataaagaagattctgtggtctaaacaaaaaaaggcggtgagcaatcctgcaagagcaaggtacataaacaagtcctcagtggttggcaactgtttcaacttgtttgaaccaagaaccttccaggaaggctaaagggaaaccgaatttcacagccatgattcttttgcccacacttgggacgaaaagattctacaaagctcttttgagcatttagactctcgactggccaaggtttggggaagaacgaacggacctttgaagaagtaaggagtcgtgtatggtagggtaagtgagagagggggatgtttcctatgctttgatcccttctcacttaacctgaagctagacgagcaggcttcttccccccaaaactgattacaactgctacagagcagacagttaagagaaatgagcttgacatttaagagaaatgagctgcactccatgagtgcagctctggaggtacgaaaagaggggaagagacttggaaatgggagacgggggcagagagggaccctccaccacctctttgggcctggctgggtgggaatgtgacttgagcccagagtgaacactcttggtagaagcccttctaccttcctgcaacacctgttccctctcagattgtaccattgagccggaa

SEQ ID NO: 8 - DNA, 240 bp, homo sapien
gtttctcctccccccggagtcagggtgtgcgaggtagggggtgtgtgtgtgtctgtgtgtgtgtgcgcggcacgtttttgagtgtgagacccaaatccagaccaggcaacctgcgttccccgcgcctccccccgtccccggtgattaatacccatttttctgacgctcccctcctcccctcctgctctccctctccggctgccgcgcggttgacttgcacagcctgcccccgcccgcttc

SEQ ID NO: 9 - DNA, 2121 bp, homo sapien
aatgcttatgtgttcgtgtgtgttaaatatgttagattcaaaaattaagcctgagaaaaaccaggaaacagaagaattactgtagctcagaagtatgagtagtctggtctacaggacagcaaaaacatatctgatggcttctacagactaaagttccaagccactaggtatatccactcacacagccttctctcctgtgcgtttgtatggccgcttctcaggaatgctggtgagaagcccatgatataaccaatatgaaaatgttccaccactgtagtattctagatatttgaaaagacaatggatgagaatgaaaatgatgcatatacaataatattaatgttaaaagtaaacaatactacaggtggtgataagggctattaaaatataaaagagttcaataggaagagaaatgaatggatgtttgccttttcaaagagggtgggtttgtaaatgccaggaaatccagtttcctttagggattattcacgggcctcaccatcttctcttctcaaggcatttaaataacaggatccactttaataaaaaatgtcaaactcctcccgccttcactagcagccagaaaagcacgcgtatggtccatactatacaatttagccacgaatgacacctgctttgctttgttttctattgtgttaggcgtcagcctcgtgtgctcagatagatcttaagctccttgaaggcaagaaagaacccggtcccagactgcgtccgacacacagaatgctcgtaggaagcacaggctgatttatggaaaatacagcaagggtcttcaatttcgaactacagctccgccttggagatgctttccgcagcccgcgggccgcccacggggggcactgtctagctcccctcgcccctcagccgctctccgtcggtgctgttccccgctgcggccgggaggaggcgccacggactcgggctaagcttcttcccccttccgtcccccttccatcccccttaccaccccacttccccagcacatgacgtgggaagctgcctccgccagagtcttagcaagaggagtgagggggtgcaggggcttgggggtggggtggggtggggggtgtggagaaagaggacacgcgcgctcacacccgcccctgggagcagaagcagtatcttccccagcttcgtgggtttctcctccccccggagtcagggtgtgcgaggtagggkgtgtgtgtgtgtctgtgtgtgtgtgcgcggcacgtttttgagtgtgagacccaaatccagaccaggcaacctgcgttccccgcgcctccccccgtccccggtgattaatacccatttttctgacgctcccctcctcccctcctgctctccctctccggctgccgcgcggttgacttgcacagcctgcccccgcccgcttcccttcccccggctccgatcctcaccaactggaatctgggctcctgcctctccaccccccacctctttttgagaaaggaaggttccccccaccccctttcaaaaaccacttcctccggcttcttcacctctaggaaatctcggggcttcgctctatcgagagtgctcgtcaagggtgattcttgatcaccaccatccagtagtgctggcgtccgaatgcatatccttttgccttgcaaagaagagtcatgaacttcaaaacccccgagacgatcgttaatcctgctttgcaggaaaggaaaaccctccattcccagccaccggccacccctctccgcagactgcaaccgcggaggcgctcggagccggaaggggacgctttgggaatgaccatgctccaccgagggacggagccggcccccagcttctccacacagagcctcctccactaacgctccaaaaaccaaaaaccgtaattgccagaagaagcgttaaaacaaaatttacgctaaattggattttaaattatcttccgttcatttatccttcgtctttcttatgtggatatgcaagcgagaagaagggactggacattcccaacatgctcactcccttaatctgtccgtctagaggtttggcttctacaaaccaagggagtcgacgagttgaagatgaagcccagagcggagtgctgttctcccaagttctggttggtgttggccg

SEQ ID NO: 10 - DNA, 6206 bp, homo sapien
taaaacaaaatttacgctaaattggattttaaattatcttccgttcatttatccttcgtctttcttatgtggatatgcaagcgagaagaagggactggacattcccaacatgctcactcccttaatctgtccgtctagaggtttggcttctacaaaccaagggagtcgacgagttgaagatgaagcccagagcggagtgctgttctcccaagttctggttggtgttggccgtcctggcggtgtcaggcagcagagctcgttctcagaagagcccccccagcattggcattgctgtcatcctcgtgggcacttccgacgaggtggccatcaaggatgcccacgagaaagatgatttccaccatctctccgtggtaccccgggtggaactggtagccatgaatgagaccgacccaaagagcatcatcacccgcatctgtgatctcatgtctgaccggaagatccagggggtggtgtttgctgatgacacagaccaggaagccatcgcccagatcctcgatttcatttcagcacagactctcacccccatcctgggcatccacgggggctcctctatgataatggcagataaggatgaatcctccatgttcttccagtttggcccatcaattgaacagcaagcttccgtaatgctcaacatcatggaagaatatgactggtacatcttttctatcgtcaccacctatttccctggctaccaggactttgtaaacaagatccgcagcaccattgagaatagctttgtgggctgggagctagaggaggtcctcctactggacatgtccctggacgatggagattctaagatccagaatcagctcaagaaacttcaaagccccatcattcttctttactgtaccaaggaagaagccacctacatctttgaagtggccaactcagtagggctgactggctatggctacacgtggatcgtgcccagtctggtggcaggggatacagacacagtgcctgcggagttccccactgggctcatctctgtatcatatgatgaatgggactatggcctccccgccagagtgagagatggaattgccataatcaccactgctgcttctgacatgctgtctgagcacagcttcatccctgagcccaaaagcagttgttacaacacccacgagaagagaatctaccagtccaatatgctaaataggtatctgatcaatgtcacttttgaggggaggaatttgtccttcagtgaagatggctaccagatgcacccgaaactggtgataattcttctgaacaaggagaggaagtgggaaagggtggggaagtggaaagacaagtccctgcagatgaagtactatgtgtggccccgaatgtgtccagagactgaagagcaggaggatgaccatctgagcattgtgaccctggaggaggcaccatttgtcattgtggaaagtgtggaccctctgagtggaacctgcatgaggaacacagccccctgccaaaaacgcatagtcactgagaataagacagacgaggagccgggttacatcaaaaaatgctgcaaggggttctgtattgacatccttaagaaaatttctaaatctgtgaagttcacctatgacctttacctggttaccaatggcaagcatgggaagaaaatcaatggaacctggaatggtatgattggagaggtggtcatgaagagggcctacatggcagtgggctcactcaccatcaatgaggaacgatcggaggtggtcgacttctctgtgcccttcatagagacaggcatcagtgtcatggtgtcacgcagcaatgggactgtctcaccttctgccttcttagagccattcagcgctgacgtatgggtgatgatgtttgtgatgctgctcatcgtctcagccgtggctgtctttgtctttgagtacttcagccctgtgggttataacaggtgcctcgctgatggcagagagcctggtggaccctctttcaccatcggcaaagctatttggttgctctggggtctggtgtttaacaactccgtacctgtgcagaacccaaaggggaccacctccaagatcatggtgtcagtgtgggccttctttgctgtcatcttcctggccagctacactgccaacttagctgccttcatgatccaagaggaatatgtggaccaggtttctggcctgagcgacaaaaagttccagagacctaatgacttctcaccccctttccgctttgggaccgtgcccaacggcagcacagagagaaatattcgcaataactatgcagaaatgcatgcctacatgggaaagttcaaccagaggggtgtagatgatgcattgctctccctgaaaacagggaaactggatgccttcatctatgatgcagcagtgctgaactatatggcaggcagagatgaaggctgcaagctggtgaccattggcagtgggaaggtctttgcttccactggctatggcattgccatccaaaaagattctgggtggaagcgccaggtggaccttgctatcctgcagctctttggagatggggagatggaagaactggaagctctctggctcactggcatttgtcacaatgagaagaatgaggtcatgagcagccagctggacattgacaacatggcaggggtcttctacatgttgggggcggccatggctctcagcctcatcaccttcatctgcgaacaccttttctattggcagttccgacattgctttatgggtgtctgttctggcaagcctggcatggtcttctccatcagcagaggtatctacagctgcatccatggggtggcgatcgaggagcgccagtctgtaatgaactcccccaccgcaaccatgaacaacacacactccaacatcctgcgcctgctgcgcacggccaagaacatggctaacctgtctggtgtgaatggctcaccgcagagcgccctggacttcatccgacgggagtcatccgtctatgacatctcagagcaccgccgcagcttcacgcattctgactgcaaatcctacaacaacccgccctgtgaggagaacctcttcagtgactacatcagtgaggtagagagaacgttcgggaacctgcagctgaaggacagcaacgtgtaccaagatcactaccaccatcaccaccggccccatagtattggcagtgccagctccatcgatgggctctacgactgtgacaacccacccttcaccacccagtccaggtccatcagcaagaagcccctggacatcggcctcccctcctccaagcacagccagctcagtgacctgtacggcaaattctccttcaagagcgaccgctacagtggccacgacgacttgatccgctccgatgtctctgacatctcaacccacaccgtcacctatgggaacatcgagggcaatgccgccaagaggcgtaagcagcaatataaggacagcctgaagaagcggcctgcctcggccaagtcccgcagggagtttgacgagatcgagctggcctaccgtcgccgaccgccccgctcccctgaccacaagcgctacttcagggacaaggaagggctacgggacttctacctggaccagttccgaacaaaggagaactcaccccactgggagcacgtagacctgaccgacatctacaaggagcggagtgatgactttaagcgcgactccgtcagcggaggagggccctgtaccaacaggtcccatatcaagcacgggacgggcgacaaacacggcgtggtcagcggggtacctgcaccttggagaagaacctgaccaacgtggagtgggaggaccggtccgggggcaacttctgccgcagctgtccctccaagctgcacaactactccacgacggtgacgggtcagaactcgggcaggcaggcgtgcatccggtgtgaggcttgcaagaaagcaggcaacctgtatgacatcagtgaggacaactccctgcaggaactggaccagccggctgccccagtggcggtgacgtcaaacgcctccaccactaagtaccctcagagcccgactaattccaaggcccagaagaagaaccggaacaaatgcgccggcagcactcctacgacaccttcgtggacctgcagaaggaagaagccgccctggccccgcgcagcgtaagcctgaaagacaagggccgattcatggatgggagcccctacgcccacatgtttgagatgtcagctggcgagagcacctttgccaacaacaagtcctcagtgcccactgccggacatcaccaccacaacaaccccggcggcgggtacatgctcagcaagtcgctctaccctgaccgggtcacgcaaaaccctttcatccccacttttggggacgaccagtgcttgctccatggcagcaaatcctacttcttcaggcagcccacggtggcgggggcgtcgaaagccaggccggacttccgggcccttgtcaccaacaagccggtggtctcggcccttcatggggccgtgccagcccgtttccagaaggacatctgtatagggaaccagtccaacccctgtgtgcctaacaacaaaaaccccagggctttcaatggctccagcaatgggcatgtttatgagaaactttctagtattgagtctgatgtctgagtgagggaacagagaggttaaggtgggtacgggagggtaaggctgtgggtcgcgtgatgcgcatgtcacggagggtgacgggggtgaacttggttcccatttgctcctttcttgttttaatttatttatggggatcctggagttctggttcctactgggggcaaccctggtgaccagcaccatctctcctccttttcacagttctctccttcttccccccgctctcagccattcctgttcccatgagatgatgccatgggtctcagcaggggagggtagagcggagaaaggaagggcagcatgcgggcttcctcctggtgtggaagagctccttgatatcctctttgagtgaagctgggagaaccaaaaagaggctatgtgagcacaaaggtagcttttcccaaactgatcttttcatttaggtgaggaagcaaaagcatctatgtgagaccatttagcacactgcttgtgaaaggaaagaggctctggctaaattcatgctgcttagatgacatctgtctaggaatcatggtccaagcagaggttgggaggccatttgtgtttatatataagccaaaaaatgcttgcttcaaccccatgagactcgatagtggtggtgaacagaacaaaaggtcattggtggcagagtggattcttgaacaaactggaaagtacgttatgatagtgtcccacggtgccttggggacaagagcaggtggattgtgcgtgcatgtgtgttcatgcacacttgcacccatgtgtagtcaggtgcctcaagagaaggcaaccttgactctttctattgtttctttcaatatccccaagcagtgtgattgtttggcttatatacagacagagatggccatgtattacctgaattttggctgtgtctcccttcatccttctggaataaggagaatgaaaattcttgataaagaagattctgtggtctaaacaaaaaaaggcggtgagcaatcctgcaagagcaaggtacataaacaagtcctcagtggttggcaactgtttcaacttgtttgaaccaagaaccttccaggaaggctaaagggaaaccgaatttcacagccatgattcttttgcccacacttgggacgaaaagattctacaaagctcttttgagcatttagactctcgactggccaaggtttggggaagaacgaacggacctttgaagaagtaaggagtcgtgtatggtagggtaagtgagagagggggatgtttcctatgctttgatcccttctcacttaacctgaagctagacgagcaggcttcttccccccaaaactgattacaactgctacagagcagacagttaagagaaatgagcttgacmtttaagagaaatgagctgcactccatgagtgcagctctggaggtacgaaaagaggggaagagacttggaaatgggagacgggggcagagagggaccctccaccacctctttgggcctggctgggtgggaatgtgacttgagcccagagtgaacactcttggtagaagcccttctaccttccygcaacacctgttccctctcagattgtaccattgagccggaa

(19) United States
(12) Patent Application Publication
(10) Pub. No.: US 2007/0048767 A1
(43) Pub. Date: Mar. 1, 2007

(54) Marker for a Psychosis or a Mood Disorder

(76) Inventors: Livia Martucci, London (GB); James Lowery Kennedy, Toronto (CA)

Correspondence Address: SUZANNAH K. SUNDBY, SMITH, GAMBRELL & RUSSEL, LLP, 1850 M STREET NW #800, WASHINGTON, DC, 20036, US

(21) Appl. No.: 11/467,272

(22) Filed: Aug. 25, 2006

Related U.S. Application Data
(60) Provisional application No. 60/710,878, filed on Aug. 25, 2005.

(51) Int. Cl. C12Q 1/68

(52) U.S. Cl. 435/6

(57) ABSTRACT

An association of a genetic marker with psychosis, a mood disorder, or a combination thereof is provided. Polymorphisms in the GRIN2B gene or alteration in the levels of GRIN2B gene products can be used to diagnose or identify a susceptibility to psychosis, a mood disorder, or a combination thereof. In certain examples, polymorphisms in the 3′UTR of the GRIN2B can be associated with psychosis, a mood disorder, or psychosis and a mood disorder. Also provided are kits for determining the presence or absence of such polymorphisms.

### CROSS REFERENCE TO RELATED APPLICATIONS

The present invention claims the benefit of U.S. Provisional Application Ser. No. 60/710,878, filed 25 Aug. 2005, which is herein incorporated by reference in its entirety.

### FIELD OF INVENTION

The present invention relates to diagnosis or identifying a risk of developing psychosis, a mood disorder, or both psychosis and a mood disorder. More particularly, the present invention relates to association of a genetic marker with psychosis, a mood disorder, or both psychosis and a mood disorder.

### BACKGROUND OF THE INVENTION

Mood disorders, for example, bipolar disorder or schizophrenia are common, chronic mental illnesses. Bipolar disorder manifests primarily as a disturbance of mood, sometimes accompanied by psychotic symptoms, whereas the core features of schizophrenia are psychosis and cognitive impairment.

The N-methyl-D-aspartate receptor (NMDAR) has been hypothesized to play a crucial role in the pathophysiology of both psychotic symptoms and disease progression in schizophrenia. The ability of NMDAR antagonists to induce a syndrome closely resembling schizophrenia suggests that dysfunction or dysregulation of NMDAR-mediated neurotransmission could play a role in schizophrenia. Several investigators have examined mRNA expression patterns for individual NMDA receptor subunits, including NR2B, in schizophrenia with somewhat conflicting results (Akbarian et al. 1996; Grimwood et al. 1999). These findings suggest that further investigation of the role of NR2B in schizophrenia is needed.

In bipolar disorder, lithium and valproate are the most well established mood stabilizers used for long-term treatment. Both of these drugs have a neuroprotective effect through reducing NMDAR-induced excitotoxicity, suggesting that alterations of glutamatergic transmission and/or of NMDARs may be involved in the symptoms of bipolar disorder.

The gene encoding Glutamate Receptor Ionotropic, N-methyl-D-aspartate 2B (GRIN2B), a subunit of NR2B, has been localized to chromosome 12p12. The full length GRIN2B cDNA has been cloned and sequenced in mouse, and has 90% homology with the human gene sequence (Schito et al. 1997). Previous attempts to detect an association between GRIN2B and schizophrenia have produced inconsistent results (Nishiguchi et al. 2000; Ohtsuki et al. 2001, Di Maria et al, 2004). More specific phenotypes have been studied in association with GRIN2B polymorphisms: a positive association was found between the C2664T polymorphism and higher clozapine dosage in 100 Chinese treatment refractory patients (Hong et al. 2001). These results were replicated in another sample consisting of 193 treatment-refractory schizophrenic patients and 176 normal subjects (Chiu et al. 2003).

Miyatake et al (2002) studied a T-to-G variant at nucleotide position −200 of the 5′UTR of GRIN2B. This substitution shortens the dinucleotide repeat to: (GT)6(CT)(GT)6, and alters the putative Sp1 binding site. Luciferase reporter assays with transfected cell-lines demonstrated that the G-variant is associated with lower gene activity (Miyatake et al. 2002). A comparison between 100 Japanese schizophrenics and 100 Japanese controls showed that the frequency of the G allele was significantly higher in schizophrenics.

In the rat and human, the NR2B subunit is primarily expressed in forebrain structures, such as the cortex, hippocampus, striatum, thalamus, and olfactory bulb. Most of the studies on changes in expression levels of NMDAR subunits have focused on the subunit NR1 (encoded by the GRIN1 gene). A higher level of binding to NR1/NR2B receptors has been reported in superior temporal cortex in schizophrenia (Grimwood et al. 1999). Gao et al (2000) analyzed postmortem hippocampal tissue from schizophrenia and healthy individuals and showed that NMDAR mRNA levels for NR1 were lower, and for NR2B higher in schizophrenia, in several hippocampal subregions (Gao et al. 2000).

In bipolar disorder, there has been relatively little investigation of the hippocampal glutamatergic system. Law and Deakin (2001) report a decrease in NR1 mRNA in subjects with bipolar disorder. While Benes et al. (2001) showed no change in the low affinity kainate receptor subunits in subjects with bipolar disorder. A third study reported a decrease of activated hippocampal NMDAR but no change in the expression of kainate or AMPA in eight subjects with Bipolar Disorder (Scarr et al. 2003).

There is a need to further clarify the association between psychosis, mood disorders and GRIN2B or GRIN2B variants.

### SUMMARY OF THE INVENTION

The present invention relates to diagnosis or identifying a risk of developing psychosis, a mood disorder, or both psychosis and a mood disorder. More particularly, the present invention relates to association of a genetic marker with psychosis, a mood disorder, or both psychosis and a mood disorder.

It is an object of the invention to provide an improved method of diagnosing psychosis, a mood disorder, or psychosis and a mood disorder, or identifying a risk of developing psychosis, a mood disorder, or psychosis and a mood disorder based on testing of the GRIN2B gene or related gene products.

According to the present invention there is provided a method (A) of diagnosing or identifying susceptibility of a subject to a mood disorder comprising, testing a sample obtained from the subject for the presence of a polymorphism in the NMDAR subunit gene GRIN2B, wherein the presence of allele C of the T/C polymorphism at nucleotide position 5988 indicates that the patient is susceptible to a mood disorder. A non-limiting example of a mood disorder is Bipolar Disorder.

The present invention also provides a method (B) of diagnosing or identifying susceptibility of a subject to psychosis comprising, testing a sample obtained from the subject for the presence of a polymorphism in the NMDAR subunit gene GRIN2B, wherein the presence of allele C of A/C polymorphism at nucleotide position 5806 indicates that the patient is susceptible to psychosis. Furthermore, psychosis is a set of symptoms that may be associated with a variety of illnesses including, but not limited to, Schizophrenia, Alzheimer's Disease, Major Depressive Disorder, Schizoaffective Disorder, or Bipolar Disorder.

The present invention also pertains to a method (C) of diagnosing or identifying susceptibility of a subject to a mood disorder comprising, testing a sample obtained from the subject for the presence of a haplotype in the NMDAR subunit gene GRIN2B, wherein the combined presence of allele T of T/G polymorphism at the nucleotide position −200 (minus 200), allele C of A/C polymorphism at nucleotide position 5806, and allele C of the T/C polymorphism at nucleotide position 5988 indicates that the patient is susceptible to a mood disorder. A non-limiting example of a mood disorder is Bipolar Disorder.

The present invention also provides a kit comprising one or more nucleic acid primers to amplify a nucleotide region, the nucleotide region comprising the putative T5988C polymorphism; one or more nucleic acid primers to amplify a nucleotide region, the nucleotide region comprising the putative A5806C polymorphism; one or more nucleic acid primers to amplify a nucleotide region, the nucleotide region comprising the putative G−200T polymorphism; one or more nucleic acid probes of between about 9 and 100 nucleotides that hybridize to nucleotide sequences comprising the T5988C polymorphism, preferably including at least one, more preferably 3 or more nucleotides both upstream and downstream of the polymorphism; one or more nucleic acid probes of between about 9 and 100 nucleotides that hybridize to nucleotide sequences comprising the A5806C polymorphism, preferably including at least one, more preferably 3 or more nucleotides both upstream and downstream of the polymorphism; one or more nucleic acid probes of between about 9 and 100 nucleotides that hybridize to nucleotide sequences comprising the A5806C polymorphism, preferably including at least one, more preferably 3 or more nucleotides both upstream and downstream thereof, one or more reagents including, but not limited to buffer(s), dATP, dTTP, dCTP, dGTP, DNA polymerase(s), instructions for diagnosing or identifying the susceptibility of a subject to psychosis or a mood disorder, instructions for using any component in the kit, or any combination thereof.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a schematic diagram of the location of polymorphisms in GRIN2B in accordance with an embodiment of the present invention.

FIG. 2 shows a representative nucleotide sequence of GRIN2B. The position of the A5806C and T5988C polymorphic sites are underlined and shown in bold.

FIG. 3 shows a representative nucleotide sequence of a 5′UTR of GRIN2B. The position of the G−200T polymorphic site is underlined and shown in bold.

FIG. 4 shows an alignment of a GRIN2B promoter region (5′UTR) as provided by Miyataki et al, 2002 (GenBank accession number AB064526) with a GRIN2B sequence comprising a partial 5′UTR, coding region and a 3′UTR (HSU88963). The locations of the polymorphic sites as described by the present invention are identified. The underlined and shaded nucleotides represent a unique DNA context sequence associated with the SNP. The italicized and shaded nucleotides (ATG) in the aligned region indicates the start codon as per Miyatake et al (2002).

### DETAILED DESCRIPTION

The present invention relates to diagnosis or identifying a risk of developing psychosis, a mood disorder, or both psychosis and a mood disorder. More particularly, the present invention relates to association of a genetic marker with psychosis, a mood disorder, or both psychosis and a mood disorder.

The following description is of a preferred embodiment.

The present invention provides a genetic marker that may be used to diagnose a mood disorder or identify a susceptibility to a mood disorder. As described in more detail below, specific polymorphisms in the GRIN2B gene may be used as an indicator of a mood disorder, for example, but not limited to bipolar disorder. Additionally, altered levels of GRIN2B mRNA or altered levels of NR2B protein may be used as an indicator of a mood disorder, for example, but not limited to bipolar disorder. Furthermore, a genetic marker is provided that may be used to diagnose psychosis or identify susceptibility to psychosis. Polymorphisms in the GRIN2B gene, altered levels of GRIN2B gene products, or a combination thereof, may be used as an indicator for psychosis.

In examples of the present invention a subject's GRIN2B gene or related gene products is assayed or tested to diagnose a mood disorder or identify a susceptibility to a mood disorder. An example of a mood disorder includes, without limitation, bipolar disorder, major depressive disorder (unipolar depression), dysthymia, cyclothymia, or depression co-morbid with another illness.

In certain examples of the present invention a subject's GRIN2B gene or related gene products is assayed or tested to diagnose psychosis or identify a susceptibility to psychosis. Psychosis is a set of symptoms that may be associated with a variety of illnesses including, but not limited to, Schizophrenia, Alzheimer's Disease, Major Depressive Disorder, Schizoaffective Disorder, or Bipolar Disorder. However, Schizophrenia does not always present with symptoms of psychosis (see Diagnostic and Statistical Manual of Mental Disorders, Fourth Edition, Washington, D.C., American Psychiatric Association, 1994, which is herein incorporated by reference).

In certain examples, specific polymorphisms in the GRIN2B gene are used as an indicator of psychosis, a mood disorder, or psychosis and a mood disorder. In other examples, altered levels of GRIN2B mRNA are used as an indicator. In still other examples, altered levels of NR2B protein are used as an indicator.

The results of assaying the GRIN2B gene or related gene products may be used alone or in conjunction with other clinical tests. In one example, a susceptibility to Bipolar Disorder can be identified by assaying for a T5988C polymorphism in the 3′ UTR of the GRIN2B gene. In another example, susceptibility to psychosis may be identified by assaying for an A5806C polymorphism in the 3′UTR of the GRIN2B gene. In still another example, results of a clinical psychiatric test, such as without limitation SCID (Structured Clinical Interview for Diagnostic and Statistical Manual Diagnosis) or FIGS (Family Interview for Genetic Studies), may be considered in conjunction with the results of assaying for a GRIN2B polymorphism.

Any tissue sample may be used for genotyping GRIN2B polymorphisms, including but not limited to, saliva or blood. In certain examples, blood is obtained from a subject for assaying with respect to GRIN2B polymorphisms. In an example, venous blood is obtained from a subject using standard venipuncture techniques.

A subject's DNA is assayed for GRIN2B polymorphisms DNA. The method of obtaining and analyzing DNA is not critical to the present invention and any methods may be used (e.g. Ausubel, et al. (eds), 1989, Current Protocols in Molecular Biology, Green Publishing Associates, Inc., and John Wiley & Sons, Inc., New York, at p. 2.10.3, or Maniatis et al., in Molecular Cloning (A Laboratory Manual), Cold Spring Harbor Laboratory, 1982, p. 387-389). For example, which is not to be considered limiting in any manner, DNA may be extracted using a non-enzymatic high-salt procedure (Lahiri and Nurnberger 199 1). Alternatively, the DNA may be analyzed in situ. Other methods of DNA analysis that are known to persons skilled in the art may also be used.

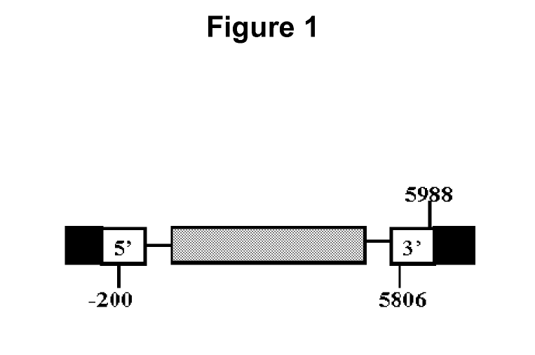

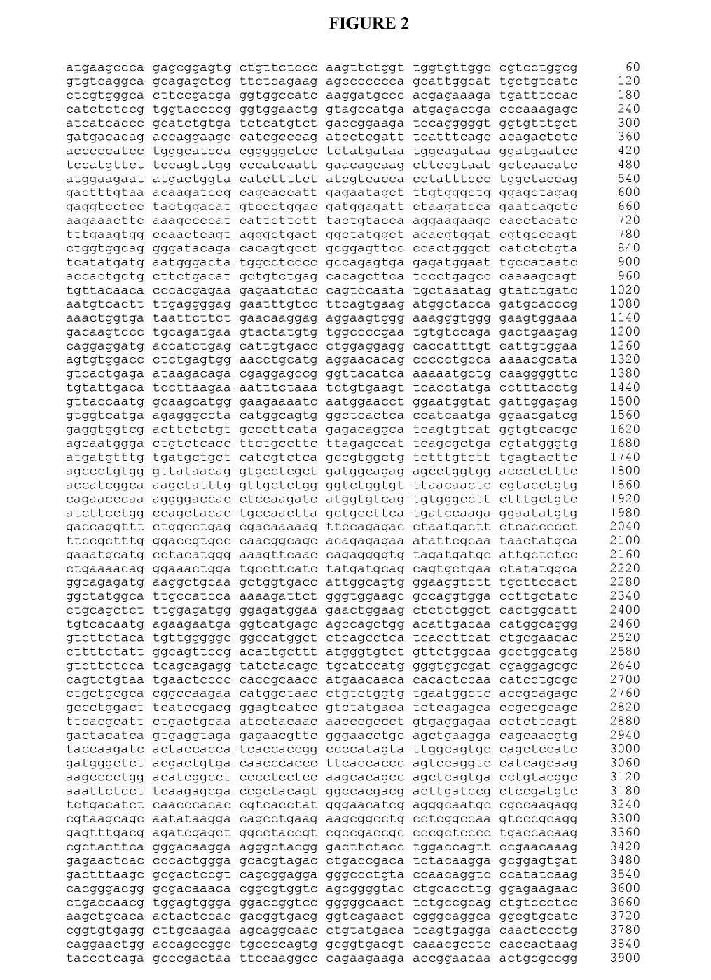

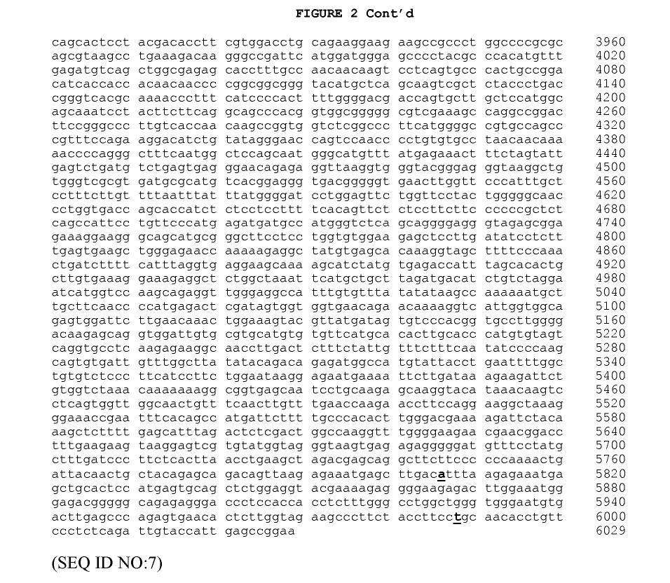

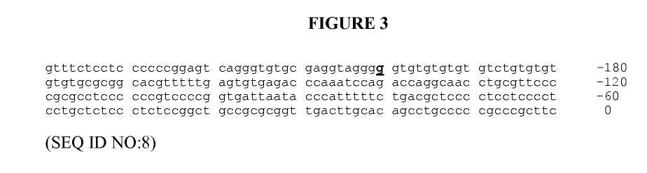

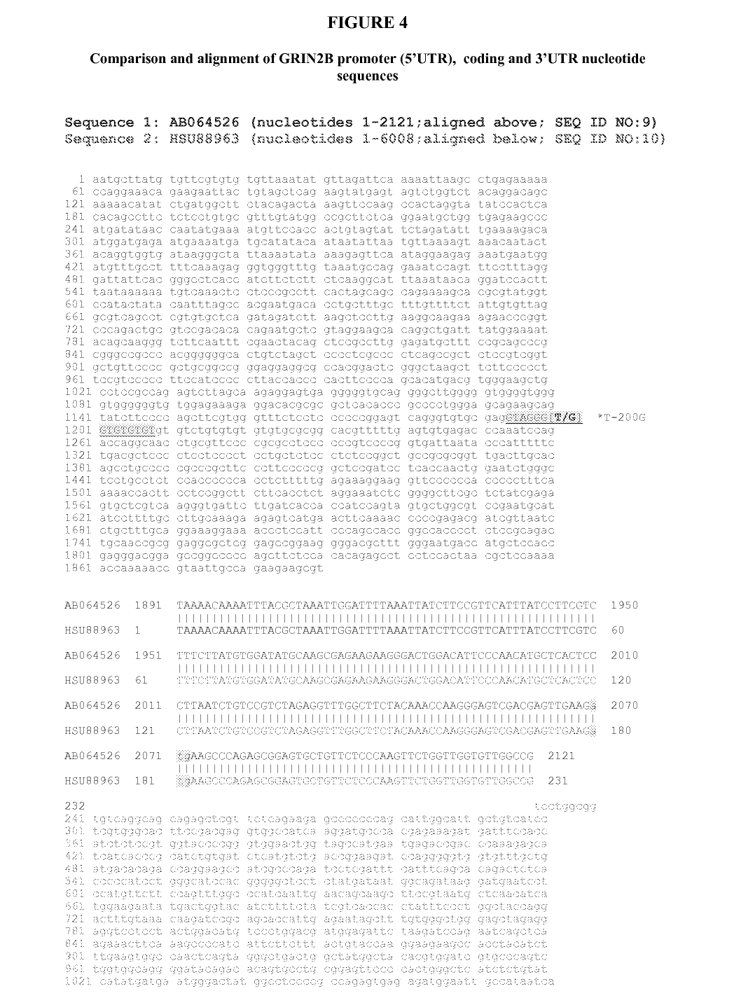

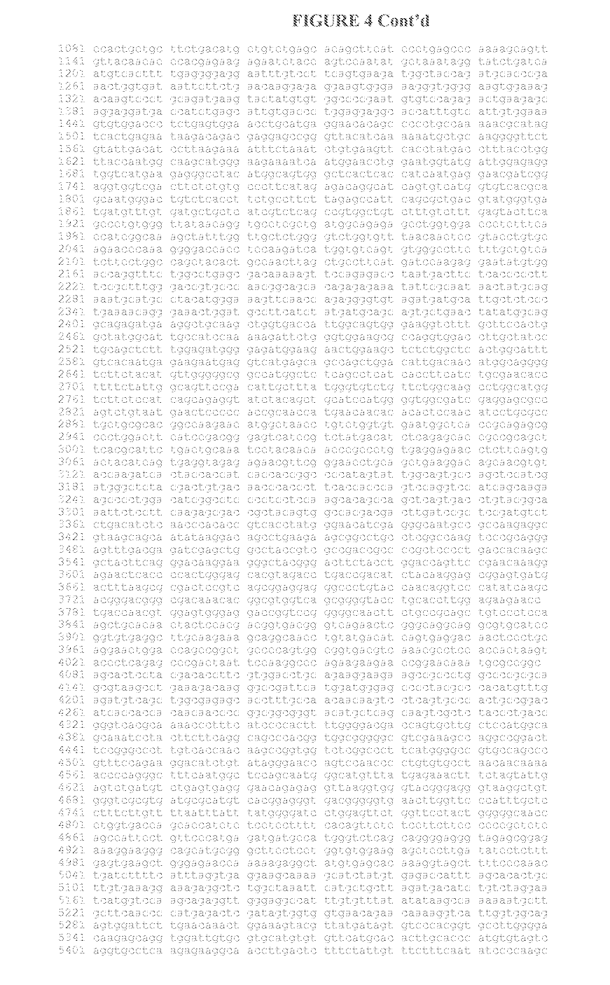

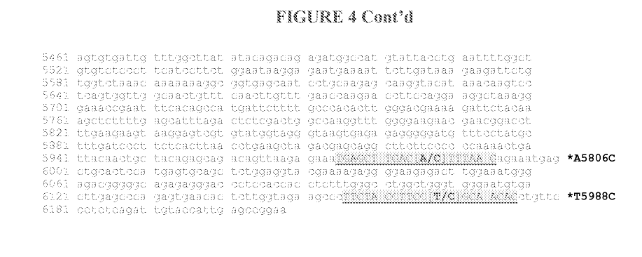

With reference to examples pertaining to assaying GRIN2B polymorphisms, examples of single nuclear polymorphism (SNP) indicators are at position −200G/T located in the 5′ UTR of GRIN2B, at position 5806A/C or 5988T/C located in the 3′ UTR of GRIN2B (see FIG. 1). The location of the polymorphisms and relative nucleotide sequences flanking the polymorphic sites is shown in FIGS. 2 and 3, respectively. As it is known in the art that various DNA numbering systems exist for genes, and because previously known gene and promoter sequences may be updated from time to time to include further sequence information, the recitation of the exact nucleotide position of a polymorphism is not meant to be absolute and could vary. Accordingly, the specific recitation of an exact position of a polymorphism is not meant to be limiting in any manner. Rather, as would be appreciated by a person of skill in the art, it is the presence or absence of a polymorphism relative to the adjacent upstream and downstream nucleotide sequences which can be used in determining risk for psychosis and/or mood disorders as described herein. In this way any nucleotide or gene sequence encoding a GRIN2B may be assayed to determine the presence or absence of one or more polymorphisms in the 5′ and 3′ UTRs of the sequence. For example, FIG. 4 shows an alignment of a GRIN2B promoter region (5′UTR) as provided by GenBank accession number AB064526 (submitted Jun. 30, 2001) with a GRIN2B sequence comprising a partial 5′UTR, coding region and a 3′UTR (HSU88963). The locations of the polymorphic sites as described by the present invention are identified therein. The underlined and shaded nucleotides represent a unique DNA context sequence associated with the SNP. The italicized and shaded nucleotides in the aligned region indicates the start codon. It may be observed that the immediately adjacent flanking sequences of the respective polymorphisms shown in FIGS. 2-4 are the same.

In a preferred embodiment, which is not meant to be limiting in any manner, at least about 15 nucleotides upstream and downstream of a polymorphism in a known UTR of a GRIN2B gene is used to determine if the polymorphism is present. More preferably at least about 17, for example, but not limited to 17, 18, 19, 20, 21, 25, 30, 50, 100 or more nucleotides upstream and downstream of a potential polymorphic site may be used, for example, but not limited to as provided in FIGS. 2, 3 and 4.

Polymorphisms may be genotyped using conventional techniques. For example, PCR using primers incorporating fluorescent probes is one suitable technique. For example, which is not to be considered limiting, primers having the following sequences:
forward primer: 5′-TCGCAGGCACTATTCTAACTACTTTAC (SEQ ID NO:1)
reverse primer: 3′GCATTCCTGAAAAGAGAGATCATGTG (SEQ ID NO:2)
may be used for the G−200T marker;
   for the A5806C marker the following sequences may be used:
forward primer: 5′-GCTACAGAGCAGACAGTTAAGAGAA (SEQ ID NO:3)
reverse primer: 3′TCATGGAGTGCAGCTCATTTCT (SEQ ID NO:4); and

for the T5988C marker, the following sequences may be used:

| | |
|---|---|
| forward primer: | |
| 5′-CTTGAGCCCAGAGTGAACACT | (SEQ ID NO:5) |
| reverse primer: | |
| 3′ACCCTCATCCCTGGAGTTTTATACA. | (SEQ ID NO:6) |

A sample from a subject can be assayed for comparing or quantifying GRIN2B mRNA levels, NR2B protein, or both GRIN2B mRNA levels and NR2B protein levels. Samples may be obtained from a variety of tissue sources. For example, in post-mortem analysis brain tissue, such as without limitation, hipoocampus, superior temporal cortex, or dorsolateral prefrontal cortex (Brodmann's area 46) may be assayed. In living subjects, if brain tissue is unavailable then other samples such as white blood cells can be assayed.

Expression levels of GRIN2B mRNA may be measured using any standard technique, for example without limitation, Northern analysis, quantitative PCR using Cyclophilin A levels as a control comparison and calculating expression levels of GRIN2B as a ratio of GRIN2B/cyclophilin threshold cycle (Tc) values, and the like.

Levels of NR2B protein may also be measured using any variety of techniques known to the skilled person, for example without limitation, ELISA, immunodiffusion, or other methods that are known to one of skill in the art.

The subject may be a human or an animal subject. For example, other mammals that may be tested include, but are not limited to a dog, cat, horse, mouse, rat, or cow. Further, human subjects may be of any ethnicity for example, but not limited to Caucasian, Asian, African American, and the like. In a preferred embodiment, which is not meant to be limiting in any manner, the subject is a Caucasian subject.

The invention also contemplates a kit comprising one or more components to diagnose or identify the susceptibility of a subject to psychosis or a mood disorder. The kit may comprise:
1) a) one or more nucleic acid primers to amplify a nucleotide region, the nucleotide region comprising the putative T5988C polymorphism;
   b) one or more nucleic acid primers to amplify a nucleotide region, the nucleotide region comprising the putative A5806C polymorphism;
   c) one or more nucleic acid primers to amplify a nucleotide region, the nucleotide region comprising the putative G−200T polymorphism,
   or a combination thereof;
2) a) one or more nucleic acid probes that hybridize to nucleotide sequences comprising the T5988C polymorphism preferably including at least one, more preferably 3 or more nucleotides upstream and downstream thereof,
   b) one or more nucleic acid probes that hybridize to nucleotide sequences comprising the A5806C polymorphism preferably including at least one, more preferably 3 or more nucleotides upstream and downstream thereof;
   c) one or more nucleic acid probes that hybridize to nucleotide sequences comprising the A5806C polymorphism preferably including at least one, more preferably 3 or more nucleotides upstream and downstream thereof,
   or a combination thereof;
3) one or more reagents including, but not limited to buffer(s), dATP, dTTP, dCTP, dGTP, DNA polymerase(s), or any combination thereof,
4) instructions for diagnosing or identifying the susceptibility of a subject to psychosis or a mood disorder;
5) instructions for using any component in the kit, or any combination or sub-combination of 1-5.

The nucleic acid primers and probes may be of any suitable length for use in the methods of the present invention. Without wishing to be limiting in any manner, it is generally preferred that the primers and probes be between about 9 and about 100 nucleotides, for example, but not limited to about 9, 11, 13, 15, 17, 19, 21, 23, 25, 27, 29, 30, 35, 40, 45, 50, 60, 70, 80, 90, about 100 nucleotides or any amount therebetween. The length of the primers and probes may also be defined by a range of any two of the values provided above. With respect to probes, it is generally preferred that the probe comprise at least one, more preferably 3 or more nucleotides on each side of the polymorphic site. It is also contemplated that one or more of the primers or nucleic acid probes may be labeled as is known in the art, for example, but not limited to, with a radioactive element or tag, fluorophore, or the like.

The present invention will be further illustrated in the following examples.

### EXAMPLES

### Example 1

### Genotyping of Living Subjects

Subjects were recruited with fully informed written consent, and in accordance with University of Toronto and Canadian Institutes of Health Research (CIHR) guidelines for the ethical treatment of human subjects.

A total of 86 nuclear families consisting of probands with schizophrenia and at least one first degree relative were collected from hospitals in Toronto, Ontario. In addition, 192 schizophrenia case-control pairs were recruited. All patients had an independent clinical DSMIIIR/DSM-IV diagnosis of schizophrenia from their referring psychiatrist. (American Psychiatric Association 1994) A SCID (Structured Clinical Interview for DSM diagnosis) was administered by trained research assistants to each proband to confirm a DSM-IIIR diagnosis of schizophrenia. After review of all clinical information, consensus between two experienced psychiatrists was the final decision for diagnosis. Controls were screened using the FIGS (Family Interview for Genetic Studies) and screening questions from the SCID, and excluded if there was any personal or family history of major mental illness or alcohol/substance abuse. Patients and control subjects were matched for age +/−5 years, sex, and self-reported ethnicity to reduce the potential stratification that might result from ethnically heterogeneous case and control groups. The average age was 37 years; with a male: female ratio of 162:180; greater than 95% of the subjects were Caucasian. The case-control sample is 100% Caucasian, so as to avoid issues of population stratification.

Venous blood was obtained from subjects using standard venipuncture techniques. DNA was extracted using a non-enzymatic high-salt procedure (Lahiri and Nurnberger 1991).

The bipolar trio sample consisted of 318 nuclear families from the Toronto area. The SCID interview and the same diagnostic procedures were used as above. The average age of the probands was 41; the male:female ratio was 565:479; and greater than 95% were Caucasian. The sample is further divided in two groups: triads with psychotic probands (N=158) and triads with non-psychotic probands (N=160).

Three SNP markers in the GRIN2B gene were studied: G−200T, A5806C and T5988C. The G−200T marker is located in the 5′UTR, whereas the A5806C and the T5988C markers are located in the 3′UTR (see FIG. 1). The A5806C marker and T5988C are in partial linkage disequilibrium. All three polymorphisms were in Hardy-Weinberg equilibrium.

Polymorphisms were genotyped using fluorescent TaqMan® probes as part of commercial Assays-on-Demand™ SNP Genotyping assays on the ABI PRISM 7000 sequence detection system, according to the manufacturer's protocol (Applied Biosystems Inc., Foster City, Calif.). The primer sequences were used:

for the G−200T marker (ABI catalogue # 4332072):

| | |
|---|---|
| Forward primer: | |
| 5′-TCGCAGGCACTATTCTAACTACTTTAC | SEQ ID NO:1 |
| Reverse primer: | |
| 3′GCATTCCTGAAAAGAGAGATCATGTG; | (SEQ ID NO:2) |

for the A5806C marker (ABI catalogue # 4332072):
forward primer: 5′-GCTACAGAGCAGACAGTTAAGAGAA (SEQ ID NO:3)
reverse primer: 3′TCATGGAGTGCAGCTCATTTCT (SEQ ID NO:4); and

for the T5988C marker (ABI catalogue # 4332072):

| | |
|---|---|
| forward primer: | |
| 5′-CTTGAGCCCAGAGTGAACACT, | (SEQ ID NO:5) |
| reverse primer: | |
| 3′ACCCTCATCCCTGGAGTTTTATACA. | (SEQ ID NO:6) |

The PCR protocol was as follows: DNA 1 μl; TaqMan MasterMix 5 μl; Assay 0.25 μl; dH20 3.75 μl. Cycling conditions were as follows: after 10 min at 95° C., the samples were submitted to 40 cycles, each consisting of a step at 95° C. for 15 s, followed by a step at 60° C. for 1 min. The PCR product was detected as an increase in fluorescence during the PCR extension phase when the probe was cleaved by the 5′ exonuclease activity of the Taq DNA polymerase. This cleavage interrupts the fluorescence resonance energy transfer and the reporter dye starts to fluoresce in proportion to the level of PCR product generated.

The informative polymorphisms and the haplotype distribution were tested in the schizophrenia and bipolar nuclear family samples using the family based association tests (FBAT; Laird et al. 2000). Differences in the allele frequencies between the patients and healthy controls were tested using the chi-square association test. Haplotype distributions in our case-control sample were obtained using Cocaphase.

Markers were tested for association with bipolar disorder in a sample of 318 nuclear families composed of father, mother and bipolar proband. The size of this bipolar sample allowed subdivision and testing of the markers for association with the specific subphenotype of psychotic symptoms in the proband.

The bipolar nuclear families were tested using FBAT; no significant association was found between the GRIN2B gene −200T/G marker and the entire bipolar sample, or with the subphenotype samples, that is, presence of psychotic symptoms (See table 1A,B).

TABLE 1A
Chi-square, and FBAT results for Case Control and schizophrenia (SCZ) triad samples
for all markers. (S): observed transmission; E(S): expected transmission.

| | T-200G | A5806C | T5988C |
|---|---|---|---|
| Case-Control, | χ² = 17.974; p = 0.0003 | χ² = 2.373; p = 0.305 | χ² = 0.849; p = 0.654 |
| scz (N = 200 | | | |
| pairs) | | | |
| SCZ Triads | z = 0.775 p = 0.44 | z = 0.349; p = 0.73 | z = 0.6; p = 0.56; |
| (N = 86 | S = 26/25; | S = 15/25; | S = 25/11; |
| pedigrees) | E(S) = 26.7/27.2IT = 26 | E(S) = 15.8/24.1; | E(S) = 23.6/12.3; |
| | | IT = 18 | IT = 16 |

TABLE 1B
Chi-square, and FBAT results for the Case Control and bipolar disorder (BP) triad
samples for all markers. (S): observed transmission; E(S): expected transmission.

| | T-200G | A5806C | T5988C |
|---|---|---|---|
| BP Triads | Z = 0.898; p = 0.37 | z = 0.395; p = 0.69; | z = 2.332; p = 0.02 |
| (N = 318 | S = 163/157 | S = 140/172 | S = 60/144; |
| pedigrees) | E(S) = 167.66/153.33 | E(S) = 137.1/174.9 | E(S) = 70.7/133.2 |
| | IT = 154 | IT = 150 | IT = 98 |
| | | | After correction for mult. |
| | | | Test. p = 0.04 |
| BP Psychotic | z = 0.101; p = 0.9 | z = 2.892; p = 0.0038 | z = 1.945; p = 0.051 |
| (N = 158 | S = 80/66; | S = 60/104; | S = 27/45; |
| pedigrees) | E(S) = 76.5/69.5 | E(S) = 75.7/88.2; | E(S) = 34.9/67.1; |
| | IT = 70 | IT = 74 | IT = 48 |
| | | After correction for mult. | |
| | | Test. p = 0.008 | |

No association was found between the A5806C polymorphism and our schizophrenia samples (both triads and case-control) (Table 1A). The overall analysis of the bipolar sample with A5806C gave negative results. However, an over transmission of allele C was detected in bipolar patients with psychotic symptoms, even after correction for multiple testing (Table 1B).

For the T5988C polymorphism: no association was found between the marker and schizophrenia in case control and both nuclear family samples (Table 1A). The analysis of the bipolar sample showed a preferential transmission of allele C in the complete bipolar sample, but with either one of the subgroups the level of significance drops to a trend. The significance is lost after correction for multiple testing. (Table 1B).

Haplotype analyses was performed for these markers with results listed in tables 2 and 3. A preferential transmission of haplotype T-C-C in the bipolar sample was detected.

TABLE 2
BP sample haplotype analyses using FBAT.

| | | Al. | | | Observed | Expected |
|---|---|---|---|---|---|---|
| HAP | Haplotype | freq. | Z score | p-value | (S) | E(S) |
| H1 | T C C (2 2 1) | 0.218 | 2.411 | 0.015899 | 91.752 | 80.402 |
| H2 | T A C (1 2 1) | 0.209 | −0.995 | 0.319957 | 69.888 | 74.627 |
| H3 | G C C (2 2 2) | 0.202 | −1.103 | 0.269829 | 66.696 | 71.862 |
| H4 | G A C (1 2 2) | 0.153 | 1.326 | 0.184876 | 63.664 | 58.275 |
| H5 | T C T (2 1 1) | 0.100 | −1.742 | 0.081573 | 28.208 | 33.852 |
| H6 | G C T (2 1 2) | 0.072 | −0.758 | 0.448665 | 18.344 | 20.555 |
| H7 | T A T (1 1 1) | 0.026 | ***** | ***** | | |
| H8 | G A T (1 1 2) | 0.021 | ***** | ***** | | |

TABLE 3
SCZ trio sample haplotype analyses using FBAT.

| | | Al. | | | Observed | Expected |
|---|---|---|---|---|---|---|
| HAP | Haplotype | freq. | Z score | p-value | (S) | E(S) |
| H1 | T A C (1 1 2) | 0.239 | 1.169 | 0.242452 | 21.579 | 18.655 |
| H2 | T C C (1 2 2) | 0.218 | −0.031 | 0.975190 | 17.421 | 17.503 |
| H3 | G A C (2 1 2) | 0.200 | 0.986 | 0.324213 | 18.421 | 16.512 |
| H4 | G C C (2 2 2) | 0.172 | −0.453 | 0.650714 | 16.579 | 17.497 |
| H5 | T C T (1 2 1) | 0.096 | −1.633 | 0.102451 | 6.000 | 9.842 |
| H6 | G C T (2 2 1) | 0.075 | 0.006 | 0.995395 | 8.000 | 7.991 |

Haplotype analyses on the schizophrenia case control sample also showed significant increase of haplotype G-C-T in cases, and of haplotype T-C-C in controls (Table 4).

TABLE 4
schizophrenia case-controls sample haplotype analysis.

| | Cases | | Controls | |
|---|---|---|---|---|
| Haplotype | EM Frequency** | Haplotype | EM Frequency** | p value* |
| T A T | 0.000279 | | | |
| T A C | 0.2119 | 1, 1, 2 | 0.1782 | 0.116 |
| T C T | 0.1539 | 1, 2, 1 | 0.1229 | 0.271 |
| T C C | 0.2026 | 1, 2, 2 | 0.2751 | 0.01 |
| G A T | 0.008576 | | | |
| G A C | 0.1366 | 2, 1, 2 | 0.1978 | 0.013 |
| G C T | 0.114 | 2, 2, 1 | 0.05462 | 0.008 |
| G C C | 0.1405 | 2, 2, 2 | 0.1703 | 0.104 |

*overall p-value = 0.00516 (between cases & controls)
**EM Frequency: Estimation Maximisation algorithm.

A significant increase of the G allele of the G(−200)T polymorphism in an schizophrenia (SCZ) case-control sample; this and the SCZ triad sample combined show association of the G allele with the disease. This result was not replicated in the schizophrenia triad sample alone. However, of the two groups, the case control one is by far the largest. Therefore, the lack of replication in the triad sample could be due to lack of power or to the inherent differences between the two sampling methods, such as bias towards earlier age at onset in triad design.

The 3′UTR marker T5988C was associated with bipolar (BP) disorder in the BP triad sample, whereas the A5806C marker showed positive association with those among the bipolar patients who had psychotic symptoms. The haplotype analyses also showed preferential transmission of haplotype G-C-T in SCZ whereas haplotype T-CC was found to be protective in a SCZ case control sample but associated with bipolar disorder in a BP sample. The analysis of the case control sample showed marked differences between cases and controls in the overall haplotype frequencies in schizophrenia. Accordingly, the haplotypes may provide a stronger predictive value than a single nucleotide polymorphism by itself.

The 5′UTR thus appears to be associated with schizophrenia, whereas the 3′UTR is associated with psychosis, mood disorder, or a combination thereof

The 5′ UTR/SCZ association support a role of the promoter region of GRIN2B in the pathophysiology of SCZ. In addition, the present work and results have identified a different region of the same gene as playing a role in bipolar disorder. This suggests that the two disorders may share some elements of etiology or pathophysiology.

Refining the phenotype, including examination of symptom clusters (such as psychotic symptoms or cognitive impairment), rather than diagnostic classifications, may provide further results.

All citations are hereby incorporated by reference.

Akbarian, S., N. J. Sucher, et al. (1996). “Selective alterations in gene expression for NMDA receptor subunits in prefrontal cortex of schizophrenics.” J Neurosci 16 (1): 19-30.

Benes, F. M., M. S. Todtenkopf, et al. (2001). “GluR5,6,7 subunit immunoreactivity on apical pyramidal cell dendrites in hippocampus of schizophrenics and manic depressives.” Hippocampus 11 (5): 482-91.

Chiu, H. J., Y. C. Wang, et al. (2003). “Association analysis of the genetic variants of the N-methyl D-aspartate receptor subunit 2b (NR2b) and treatment-refractory schizophrenia in the Chinese.” Neuropsychobiology 47 (4): 178-81.

Di Maria, E., Gulli, R., et al (2004) “Variations in the NMDA Receptor subunit 2B Gene (GRIN2B) and Schizophrenia: A Case-Control Study”. American Journal of Medical Genetics Part B (Neuropsychiatric Genetics) 128B:27-29.

Diagnostic and Statistical Manual of Mental Disorders, Fourth Edition, Washington, D.C., American Psychiatric Association, 1994.

Gao, X. M., K. Sakai, et al. (2000). “Ionotropic glutamate receptors and expression of N-methyl-D-aspartate receptor subunits in subregions of 21 human hippocampus: effects of schizophrenia.” Am J Psychiatry 157 (7): 1141-9.

Grimwood, S., P. Slater, et al. (1999). “NR2B-containing NMDA receptors are up-regulated in temporal cortex in schizophrenia.” Neuroreport 10 (3): 461-5.

Hedges, L. V. a. O., I (1985). Statistical methods for meta-analysis. Orlando (Fla.), Academic Press.

Hong, C. J., Y. W. Yu, et al. (2001). “Association analysis for NMDA receptor subunit 2B (GRIN2B) genetic variants and psychopathology and clozapine response in schizophrenia.” Psychiatr Genet 11 (4): 219-22.

Lahiri, D. K. and J. I. Numberger, Jr. (1991). “A rapid non-enzymatic method for the preparation of HMW DNA from blood for RFLP studies.” Nucleic Acids Res 19 (19): 5444.

Laird, N. M., S. Horvath, et al. (2000). “Implementing a unified approach to family-based tests of association.” Genet Epidemiol 19 Suppl 1: S36-42.

Law, A. J. and J. F. Deakin (2001). “Asymmetrical reductions of hippocampal NMDAR1 glutamate receptor mRNA in the psychoses.” Neuroreport 12 (13): 2971-4.

Mandich, P., Schito, A. M., Bellone, E., Antonacci, R., Finelli, P., Rocchi, M. and Ajmar, F. (1994). “Mapping of the human NMDAR2B receptor subunit gene (GRIN2B) to chromosome 12p12.” Genomics 22 (1), 216-218 (1994); GenBank Accession submission U88963.

Miyatake, R., A. Furukawa, et al. (2002). “Identification of a novel variant of the human NR2B gene promoter region and its possible association with schizophrenia.” Mol Psychiatry 7 (10): 1101-6.

Miyatake, R., Furukawa, A. and Suwaki, H. (2002) Homo sapiens hNR2B gene, promoter region, Genbank accession number AB064526.

Nishiguchi, N., O. Shirakawa, et al. (2000). “Novel polymorphism in the gene region encoding the carboxyl-terminal intracellular domain of the NMDA receptor 2B subunit: analysis of association with schizophrenia.” Am J Psychiatry 157 (8): 1329-31.

Ohtsuki, T., K. Sakurai, et al. (2001). “Mutation analysis of the NMDAR2B (GRIN2B) gene in schizophrenia.” Mol Psychiatry 6 (2): 211-6.

Scarr, E., G. Pavey, et al. (2003). “Decreased hippocampal NMDA, but not kainate or AMPA receptors in bipolar disorder.” Bipolar Disord 5 (4): 257-64.

Schito, A. M., A. Pizzuti, et al. (1997). “mRNA distribution in adult human brain of GRIN2B, a N-methyl-D-aspartate (NMDA) receptor subunit.” Neurosci Lett 239 (1): 49-53.

The present invention has been described with regard to one or more embodiments. However, it will be apparent to persons skilled in the art that a number of variations and modifications can be made without departing from the scope of the invention as defined in the claims.

What is claimed is:

1. A method of diagnosing or identifying susceptibility of a subject to a mood disorder comprising:
testing a sample obtained from the subject for the presence of a polymorphism in the NMDAR subunit gene GRIN2B, wherein the presence of allele C of the T/C polymorphism at nucleotide position 5988 indicates that the patient is susceptible to a mood disorder.

2. The method of claim 1, wherein the sample is blood.

3. The method of claim 1, wherein the mood disorder is selected from the group consisting of Depression co-morbid with another illness, Major Depressive Disorder, Dysthymia, Cyclothymia, and Bipolar Disorder.

4. The method of claim 1, wherein the step of testing comprises DNA extraction and PCR analysis.

5. A method of diagnosing or identifying susceptibility of a subject to psychosis comprising:
testing a sample obtained from the subject for the presence of a polymorphism in the NMDAR subunit gene GRIN2B, wherein the presence of allele C of A/C polymorphism at nucleotide position 5806 indicates that the patient is susceptible to psychosis.

6. The method of claim 5, wherein the sample is blood.

7. The method of claim 5, wherein the psychosis is associated with an illness selected from the group consisting of Schizophrenia, Alzheimer's Disease, Major Depressive Disorder, Schizoaffective Disorder and Bipolar Disorder.

8. The method of claim 5, wherein the step of testing comprises DNA extraction and PCR analysis.

9. A method of diagnosing or identifying susceptibility of a subject to a mood disorder comprising:
testing a sample obtained from the subject for the presence of a haplotype in the NMDAR subunit gene GRIN2B, wherein the combined presence of allele T of T/G polymorphism at the nucleotide position −200 (minus 200), allele C of A/C polymorphism at nucleotide position 5806, and allele C of the T/C polymorphism at nucleotide position 5988 indicates that the patient is susceptible to a mood disorder.

10. The method of claim 9, wherein the sample is blood.

11. The method of claim 9, wherein the mood disorder is selected from the group consisting of Depression co-morbid with another illness, Major Depressive Disorder, Dysthymia, Cyclothymia, and Bipolar Disorder.

12. The method of claim 9, wherein the step of testing comprises DNA extraction and PCR analysis.

13. A kit comprising
1) a) one or more nucleic acid primers to amplify a nucleotide region, the nucleotide region comprising the putative T5988C polymorphism;
b) one or more nucleic acid primers to amplify a nucleotide region, the nucleotide region comprising the putative A5806C polymorphism;
c) one or more nucleic acid primers to amplify a nucleotide region, the nucleotide region comprising the putative G-200T polymorphism,
or a combination thereof;
2) a) one or more nucleic acid probes that hybridize to nucleotide sequences comprising the T5988C polymorphism including at least one, preferably 3 or more nucleotides upstream and downstream thereof,
b) one or more nucleic acid probes that hybridize to nucleotide sequences comprising the A5806C polymorphism including at least one, preferably 3 or more nucleotides upstream and downstream thereof;
c) one or more nucleic acid probes that hybridize to nucleotide sequences comprising the A5806C polymorphism including at least one, preferably 3 or more nucleotides upstream and downstream thereof,
or a combination thereof;
3) one or more reagents including, but not limited to buffer(s), dATP, dTTP, dCTP, dGTP, DNA polymerase(s), or any combination thereof,
4) instructions for diagnosing or identifying the susceptibility of a subject to psychosis or a mood disorder;
5) instructions for using any component in the kit,
or any combination of items or subitems of 1-5.
